(19) United States
(12) Patent Application Publication
(10) Pub. No.: US 2003/0142271 A1
(43) Pub. Date: Jul. 31, 2003

(54) Aberration and corneal topography measurement

(76) Inventors: Denwood F. Ross, Austinburg, OH (US); Larry G. Jone, Jacksonville, FL (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/058,739

(22) Filed: Jan. 30, 2002

(51) Int. Cl. A61B 3/10

(52) U.S. Cl. 351/212

(57) ABSTRACT

A method and apparatus for measuring with a single device both the aberrations introduced by an eye and the topography of the cornea of the eye. The method includes determining aberrations within a wavefront created by reflecting a beam off the retina of an eye, determining the corneal topography of the eye from a pattern reflected by the cornea, and directing the beam, wavefront, and reflected pattern using a combiner/separator. The apparatus includes a source for generating the beam for producing the wavefront exiting the eye and a first imaging device for receiving the wavefront to determine aberrations, a projector for projecting the pattern onto the cornea for reflection by the cornea and a second imaging device for receiving the reflected pattern to determine corneal topography, and a combiner/separator for directing the beam, wavefront, and reflected pattern.

### FIELD OF THE INVENTION

The present invention relates to ophthalmic instruments and, more particularly, to methods and apparatus for measuring both the aberrations introduced by a patient's eye and the corneal topography of the eye.

### BACKGROUND OF THE INVENTION

The eye is an optical system having several optical elements for focusing light rays representing images onto the retina within the eye. Imperfections in the components and materials within the eye and the topography of the surface of the cornea, however, may cause light rays to deviate from the desired path. These deviations, referred to as aberrations, result in blurred images and decreased visual acuity, which can be corrected by determining the aberrations and compensating for them. In addition, the topography of the cornea is indicative of certain ophthalmic disorders and its determination is necessary to make accurate refractive changes to the eye in surgical procedures such as RK, PK, or LASIK. Hence, methods and apparatus for determining aberrations introduced by an eye and the topography of the cornea of the eye are useful.

FIG. 1 is an illustration of a prior art Hartman-Shack Wavefront Measuring Device (WMD) 100 for measuring aberrations introduced by an eye 102 in a wavefront exiting the eye 102. An example of a Hartmann-Shack WMD is described in U.S. Pat. No. 5,777,719 to Williams et al., entitled Method and Apparatus for Improving Vision and the Resolution of Retinal Images, incorporated fully herein by reference.

In the WMD 100, an input beam 104 generated by a radiation source 106, e.g., a laser, is routed to the eye 102 by a beam splitter 108 where it is focused to a small spot 110 on the retina 112 within the eye 102. A wavefront 114 reflected from the spot 110 on the retina 112, which acts as a diffuse reflector, becomes aberrated as it passes through the lens and other components and materials within the eye 102 and exits through the cornea 116. In an ideal eye, the wavefront 114 would be free of aberrations. In an imperfect eye 102, however, aberrations are introduced as the wavefront 114 passes out of the eye 102, resulting in an imperfect wavefront containing aberrations.

On the return path, the wavefront 114 passes through the beam splitter 108 to an imaging device 118 that includes, for example, a Hartman-Shack lenslet array 120 and a charge coupled device (CCD) 122. A quarter-wave plate 124, positioned between the eye 102 and the beam splitter 108, is a known technique for manipulating the polarization of the input beam 104 going into the eye 102 and the wavefront 114 emanating from the eye 102 to allow the wavefront 100 to pass through the beam splitter 108 (assuming a polarized beam splitter) toward the imaging device 118. Additional lenses 126 are positioned between the eye 102 and the imaging device 118 to image the plane of the pupil of the eye 106 onto the imaging device 118 with a desired magnification. Information detected by the imaging device 118 is then processed by a processor 128 to determine the aberrations of the wavefront 114.

FIG. 2 is a cross-sectional view of a prior art Keratometer 130 for determining the topography of the cornea 116 of the eye 102. The Keratometer 130 determines the topography, i.e., curvature, of the front surface 132 of the cornea 116 by projecting a plurality of concentric rings onto the cornea 116 and, then, examining the concentric rings as reflected by the cornea 116. An example of a Keratometer 130 is described in U.S. Pat. No. 4,772,115 to Gersten et al., entitled Illuminated Ring Keratometer Device, incorporated fully herein by reference.

In the Keratometer 130, a pattern projector 133 including one or more light sources 134 and a hollow cone 136 projects concentric rings onto the surface 132 of the cornea 116. The light source 134 emits light that is channeled toward the cornea 116 by the hollow cone 136, which defines a cylindrical passageway 138. The cylindrical passageway 138 contains alternating opaque sections 140 and translucent sections 142. Light from the light source 134 reflects off the inner surface 144 of the cylindrical cone 136 and passes through the translucent sections 142 of the cylindrical passageway 138 to form concentric rings (represented by points 146) on the cornea 116, such as the concentric rings 148 depicted in FIG. 2A. The cornea 116 reflects the light of the concentric rings toward an imaging device 150, which captures the reflected concentric rings to determine the topography of the cornea 116. The reflected concentric rings, which contain information related to the topography of the cornea 116, can be read like a topographic map. When the separation between the rings is wide, the curvature, or refractive power, of the cornea 116 is less, and conversely, narrow separation between the rings indicates more curvature or higher refractive power. The information captured by the imaging device 150 can be digitized and processed by a processor 152 using image-processing techniques to determine the topography of the cornea 116.

Heretofore, WMDs 100 (FIG. 1) and Keratometers 130 (FIG. 2) have been produced as separate devices. Since separate devices are used, one of the devices is first used to make one measurement, e.g., to measure aberrations or determine corneal topography, and, then, the other device is used to make the other measurement. For example, the WMD 100 may be used to first determine the aberrations of the eye 102 and, then, the Keratometer 130 may be used to determine the topography of the cornea 116.

Using separate devices leads to inefficiencies in time, components, and storage space. Inefficiencies in time are due to the time and inconvenience required to switch between devices and to align separate devices with the eye 102 when measuring aberrations and determining corneal topographies. Also, using separate components is wasteful since each device may contain duplicate components of the other, e.g., duplicate housings and power supplies. Furthermore, separate devices require a larger “footprint” than a single device, thereby taking up a larger percentage of available space in an office. Accordingly, methods and apparatus for measuring both wavefront aberrations and corneal topography in a single device are needed. The present invention fulfils this need among others.

### SUMMARY OF THE INVENTION

The present invention provides a method and an apparatus for measuring both the aberrations introduced by an eye and the corneal topography of the cornea of the eye. A single device measures both aberrations introduced by the eye and the corneal topography of the eye. With such a single device, efficiencies in terms of time, components, and storage space are realized.

A method embodiment includes directing a beam into the eye to produce a wavefront exiting the eye along a first path. Additionally, a pattern is projected onto the surface of the cornea of the eye to produce a reflected pattern along the first path. The wavefront and the reflected pattern are directed into second and third paths, respectively. The wavefront aberrations introduced by the eye are determined, and from the reflected pattern the topography of the surface of the cornea is determined.

An apparatus embodiment includes a source for generating a beam that is capable of producing a wavefront exiting the eye and a pattern projector for projecting a pattern onto the cornea of the eye that is capable of being reflected by the cornea of the eye. A beam splitter directs the wavefront and the reflected pattern. A first imaging device receives the wavefront, and a second imaging device receives the reflected pattern.

### BRIEF DESCRIPTION OF THE DRAWINGS

FIG. 1 is schematic diagram of a prior art WMD for measuring aberrations introduced by an eye;

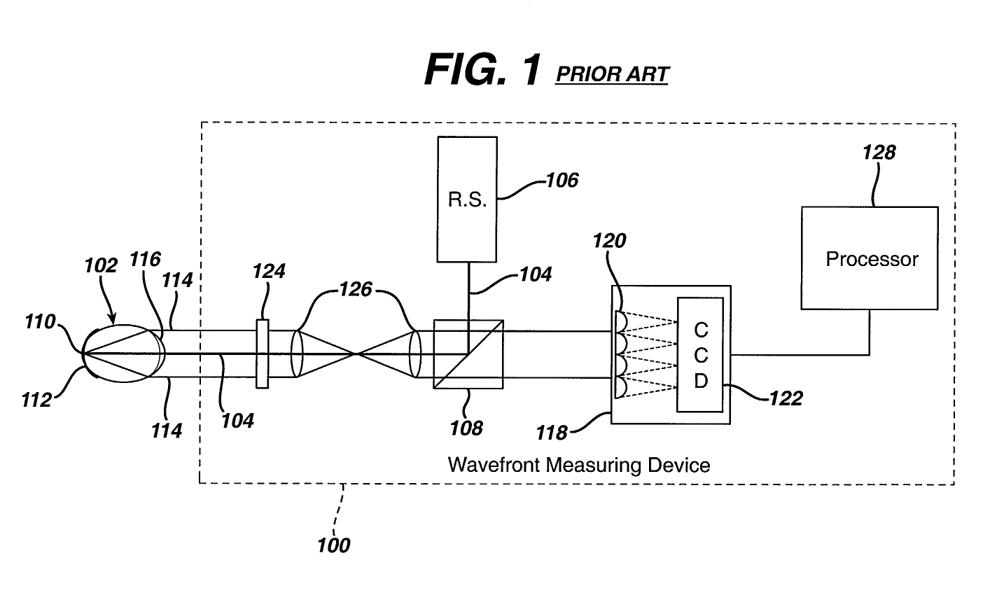

FIG. 2 is a cross-sectional view of a prior art Keratometer for determining the topography of the cornea of an eye;

FIG. 2A is an illustration of a pattern formed on the cornea of the eye using the Keratometer of FIG. 2; and

FIG. 3 is a schematic diagram of a wavefront aberration and corneal topography measurement apparatus in accordance with the present invention.

### DETAILED DESCRIPTION OF THE INVENTION

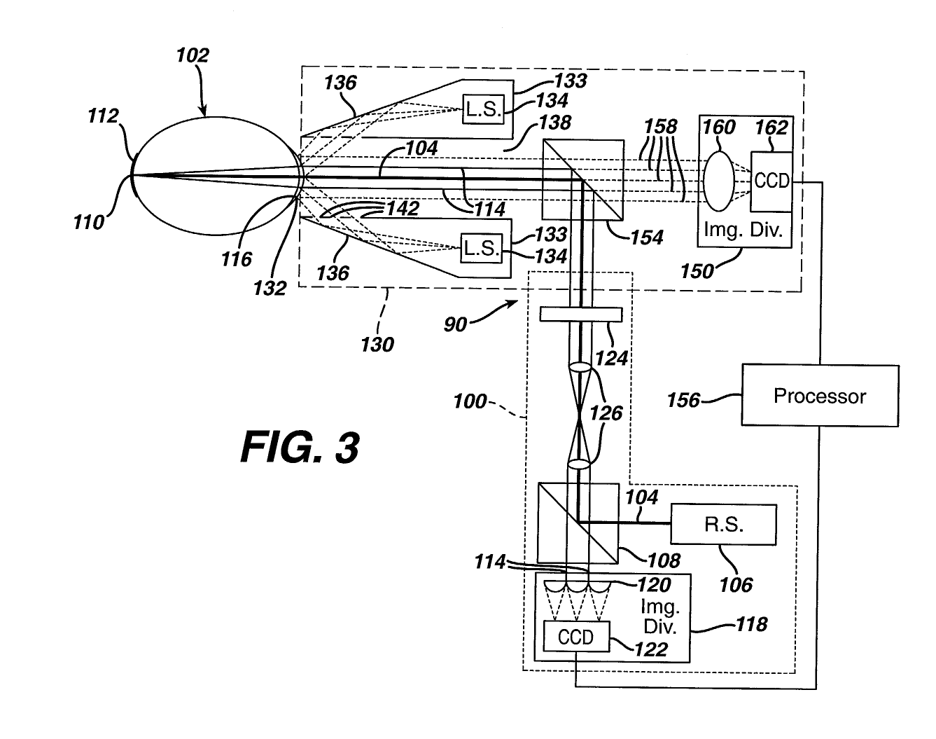

Illustrated in FIG. 3 is an embodiment of an aberration and corneal topography measurement apparatus 90 in accordance with the present invention. In a general overview, aberrations and corneal topography measurements are performed by the single device 90 to determine both the aberrations introduced by the eye 102 and the corneal topography of the cornea 116 of the eye 102. Aberrations introduced by the eye 102 are determined by directing an input beam 104 created by a radiation source 106 into the eye 102 to produce a wavefront 114 that travels back out of the eye 102. Aberrations within the wavefront 114 are then captured by a first imaging device 118 for analysis by a processor 156.

The corneal topography of the cornea 116 is determined by projecting a pattern onto the surface 132 of the cornea 116 with a pattern projector 133. A reflected pattern 158 off of the cornea 116 is then directed to a second imaging device 150 to capture the reflected pattern for analysis by the processor 156 to determine the topography of the cornea 116.

A combiner/separator 154 directs the beam 104, the wavefront 114, and the reflected pattern 158 within the device 90 as necessary for the particular function being carried out. Aberration measurement, corneal topography measurement, and the combiner/separator 154 are described in detail below.

### Aberration Measurement

In the illustrated embodiment, aberration measurements are performed by the WMD 100 depicted in FIG. 3. The WMD 100 is capable of determining aberrations introduced by the eye 102. The WMD 100 includes a radiation source 106, beam splitter 108, and a first imaging device 118. The radiation source 106 generates the input beam 104 for forming a spot 110 on the retina 112 of the eye 102. The retina 112 reflects the input beam 104 as the wavefront 114, which is aberrated as it passes out of the eye 102. The radiation source 106 may be a known laser that produces a focused beam of photons near a single frequency. In one embodiment, the single frequency is above about 700 nm, e.g., 740 nm. By choosing a frequency above the visible spectrum, i.e., above about 700 nm, the input beam 104 and resultant wavefront 114 will not cause the size of the pupil to shrink, which would limit the portion of the eye 102 for which aberrations could be determined.

The beam splitter 108 directs the input beam 104 toward the eye 102 via the combiner/separator 154, described below, and directs the wavefront 1 14 toward the imaging device 118 as shown. In the illustrated embodiment, the combiner/separator 154 reflects the input beam 104 toward the eye 102 and the resulting wavefront 114 toward the imaging device 118. In one embodiment, the beam splitter 108 is a polarized beam splitter for directing the input beam 104 and wavefront 114 based on their polarity. If a polarized beam splitter 108 is used, a ¼ wave plate 124 is provided to manipulates the input beam 104 and the wavefront 114 in a known manner such that the polarized beam splitter can direct the input beam 104 and the wavefront 114 appropriately as shown.

The imaging device 118 receives the wavefront 114 from the eye 102 and captures information related to the aberrations introduced by the eye 102. In the illustrated embodiment, the imaging device 118 includes a known Hartmann-Shack lenslet array 120 and charge coupled device (CCD) 122. The Hartmann-Shack lenslet array 120 focuses the wavefront 114 onto the CCD 122 in a known manner to produce a plurality of images on the CCD 122 that can be used to determine aberrations introduced by the eye 102.

The processor 156 receives the captured information from the imaging device 118 and processes the information using known techniques to determine aberrations introduced by the eye 102. The processor 156 may be positioned within a housing containing the aberration and corneal topography measurement apparatus of FIG. 3 or may be a separate device, e.g., a laptop computer, that can be connected to the aberration and corneal topography measurement apparatus of FIG. 3.

In use, the input beam 104 generated by the radiation source 106 is routed to the eye 102 by the beam splitter 108 and the combiner/separator 154, where it is focused to a small spot 110 on the retina 112 within the eye 102. The wavefront 114 reflected from the spot 110 on the retina 112 becomes aberrated as it passes from the retina 112 out of the eye 102. On the return path, the wavefront 114 is reflected by the combiner/separator 154, and passes through the beam splitter 108 to the imaging device 118. Information captured by the imaging device 118 is then processed by the processor 156.

### Corneal Topography Measurement

In the illustrated embodiment, corneal topography measurements are performed by the Keratometer 130 depicted in FIG. 3. The Keratometer 130 is capable of determining the topography of the front surface 132 of the cornea 116 of the eye 102. The Keratometer 130 includes a pattern projector 133 and an imaging device 150. An example of a suitable pattern projector 133 and imaging device 150 can be found in a Scout Topographer produced by Optikon 2000 of Rome, Italy and available through EyeQuip™, a division of Alliance Medical Marketing Inc. of Ponte Vedra Beach, Fla. USA.

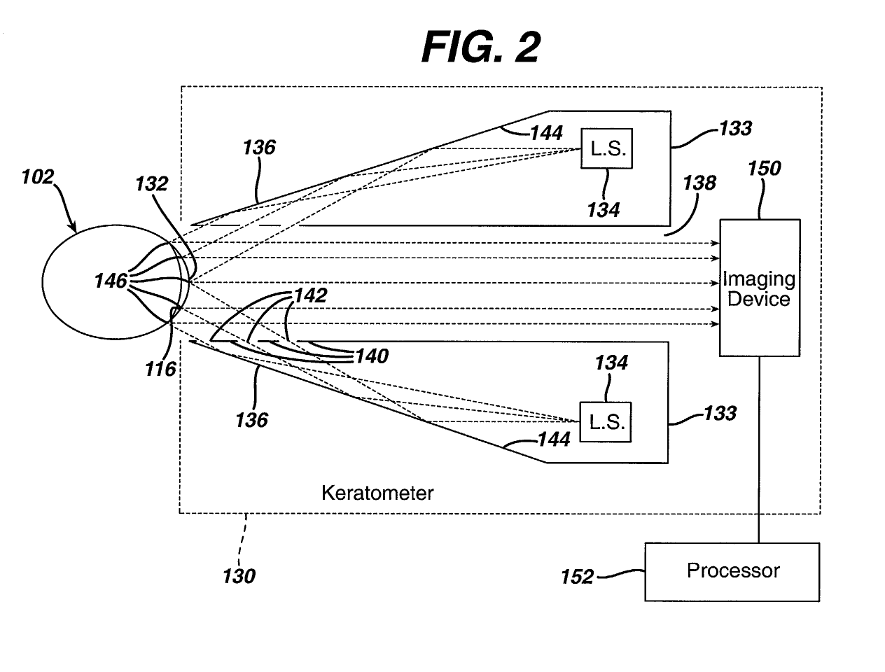

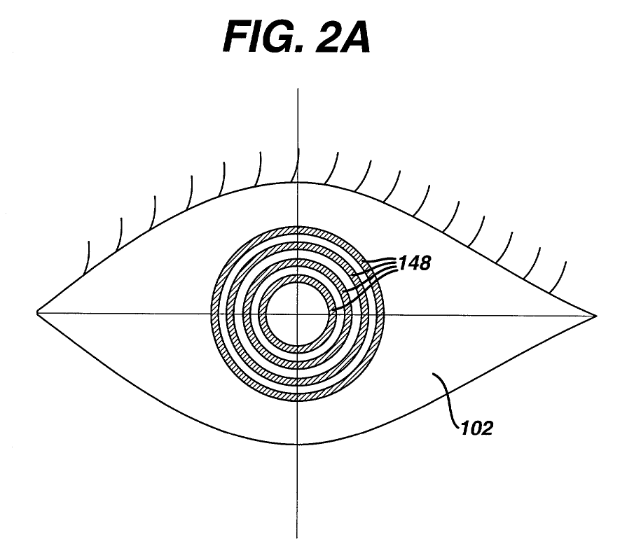

The pattern projector 133 generates an image for projection onto the cornea 116. In the illustrated embodiment, the pattern projector 133 includes a light source 134, e.g., a plurality of LEDs, and a cone 136. The cone 136 directs the light from the light source 134 to the cornea 116 via translucent sections 142 within the cone 136 to form a pattern on the cornea 116 of the eye 102 in a known manner, such as the concentric ring pattern 148 depicted in FIG. 2A. The pattern is reflected by the cornea 116 as a reflected pattern 158. In the illustrated embodiment, the cone 136 includes a cylindrical passageway 138 through the center of the cone 136 to allow the reflected pattern 158 to pass through to the imaging device 150 via the combiner/separator 154. In addition, the cylindrical passageway 138 allows the beam 104 and wavefront 114 associated with the WMD 100 to pass through. The pattern projector 133 may be a known Placido ring projector capable of projecting Placido rings, i.e., concentric rings, onto the cornea 116.

In one embodiment, the light source 134 projects light of a single wavelength below about 700 nm, e.g, 680 nm. Since the frequency is in the visible spectrum, i.e., below about 700 nm, the size of the pupil of the eye 102 may be affected by the light source 134. This does not interfere with the measurement of corneal topography, however, since the pattern produced by the light source 134 is reflected by the cornea 116 prior to passing through the pupil. In an alternative embodiment, the light source 134 projects light having a frequency above about 700 nm and, therefore, will not affect the pupil.

The imaging device 150 receives the reflected pattern 158 from the eye 102 and captures information related to the reflected pattern 158. In the illustrated embodiment, the imaging device 150 includes a known lens 160 and charge coupled device (CCD) 162. The lens 160 focuses the reflected pattern 158 onto the CCD 162 to produce an image of the reflected pattern on the CCD 162.

The processor 156 receives the captured information from the imaging device 150 and processes the information using known techniques to determine the topography of the cornea 116. In the illustrated embodiment, the processor 156 for determining the topography of the cornea 116 is the same processor for determining the aberrations introduced by the eye 102 in the WMD 100 described above. It is contemplated that a separate processor could be employed to determine the corneal topography of the eye 102.

In use, the pattern projector 133 projects an image onto the cornea 116, where it is reflected by the cornea 116 as a reflected pattern 158 containing information related to the topography of the cornea 116. The reflected pattern 158 passes through the cylindrical passageway 138 and is then directed toward the imaging device 150. In the illustrated embodiment, the combiner/separator 154, discussed below, allows the reflected pattern 158 to pass through unaffected to the imaging device 150 where information related to the topography of the cornea 116 contained within the reflected pattern 158 is captured. The captured information is then passed to the processor 156 for processing in a known manner to determine the topography of the cornea 116.

### Combiner/Separator 154

In the illustrated embodiment, the beam 104 and wavefront 114 for aberration measurement and the reflected pattern 158 (as reflected by the eye 102) for corneal topography measurement can pass along a common pathway and be appropriately directed by the combiner/separator 154. The combiner/separator 154 directs the wavefront 114 toward the imaging device 118 by reflecting the wavefront 114 and directs the reflected pattern 158 toward the imaging device 150 by allowing it to pass through the combiner/separator 154 unaffected. Also, in the illustrated embodiment, the combiner/separator 154 performs the additional function of directing the input beam 104 into the eye 102 by reflecting the input beam 104. As illustrated, the input beam 104 entering the eye 102, the wavefront 114 exiting the eye 102, and the reflected pattern 158 reflected by the eye 102 may all be on a common optical pathway, which passes through the cylindrical passageway 138 of the pattern projector 133. In an alternative embodiment, the combiner/separator 154 reflects the reflected pattern 158 and allows the input beam 104 and the wavefront 114 to pass through unaffected, the WMD 100 and Keratometer 130 being repositioned accordingly.

The combiner/separator 154 may be a dichroic mirror that passes light having a frequency below a certain “pass” level and reflects light having a frequency above the pass level. Using a dichroic mirror, the wavefront 114 and the reflected pattern 158 can be appropriately directed based on their respective frequencies. The pass level, the frequency of the input beam 104 (which generates the wavefront 114 of an equivalent frequency), and the frequency of the light source 134 (which generates the reflected pattern 158 of an equivalent frequency) are selected such that the pass level falls between the frequencies of the reflected pattern 158 and the wavefront 114. For example, if a radiation source 106 has a frequency of approximately 760 nm and the light source 134 has a frequency of approximately 680 nm, a dichroic mirror having a pass level of about 720 nm would be selected to allow the resultant wavefront 114 to be reflected and the reflected pattern 158 to pass through unaffected. The dichroic mirror is selected such that the frequency of its pass level is sufficiently different from the frequencies of the radiation source 106 and the light source 134 to accommodate “bleed through,” which occurs around the pass level.

It is contemplated that the combiner/separator 154 may be some other type of beam splitter capable of differentiating the input beam and wavefront 104, 114 from the reflected pattern 158. For example, the combiner/separator 154 may be a polarized beam splitter that differentiates based on the polarity of the input beam/wavefront 104/114 versus the polarity of the reflected pattern 158.

In use, the illustrated aberration and corneal topography measurement apparatus 90 of FIG. 3 can be used to measure aberrations introduced by the eye 102 and the corneal topography of the eye 102 in the following manner. The radiation source 106 generates an input beam 104 that is reflected, first, by the beam splitter 108 and, second, by the combiner/separator 154 toward the eye 102. A wavefront 114 produced by the eye 102 in response to the input beam 104 exits the eye 102 and is reflected by the combiner/separator 154 toward the beam splitter 108 and the imaging device 118. The wavefront 114 passes through the beam splitter 108 and strikes the imaging device 118 where information related to the aberrations introduced by the eye 102 is captured.

Separately, the pattern projector 133 projects a pattern onto the cornea 116 of the eye. The resulting reflected pattern 158 contains information related to the topography of the cornea 116. The reflected pattern 158 passes through the combiner/separator 154 and strikes the imagining device 150 where the information related to the corneal topography ic captured. The aberrations and the corneal topography of the eye 102 can then be determined by the processor 156 coupled to the imaging devices 118, 150.

The aberration and corneal topography measurement apparatus 90 may determine the aberrations of the eye 102 during one period of time and determine the corneal topography of the eye 102 during a second period of time. By separating the measurements in time, the visible wavelengths of light typically used in Keratometers 130, which may adversely affect aberration measurement by affecting the size of the pupil, will not interfere with the measurement of aberrations by the WMD 100. For example, the aberrations may be measured by the WMD 100 first to avoid being affected by the corneal topography measurement of the Keratometer 130 or a delay may be introduced after corneal topography measurement to allow the eye 102 to dilate. In an alternative embodiment, the frequencies of light used by both the WMD 100 and Keratometer 130 are outside of the visible spectrum, thereby allowing aberration and corneal topography measurements to occur substantially simultaneously.

The present invention thus provides a unique device capable of performing functions of both a WMD 100 and Keratometer 130 in a single device, which can be provided in a common housing having a small form factor such as a handheld device.

Having thus described a few particular embodiments of the invention, various alterations, modifications, and improvements will readily occur to those skilled in the art. For example, it is contemplated that the wavefront 114 and the reflected pattern 158 may be routed such that a single imaging device could be used to capture information from both the wavefront 114 and the reflected pattern 158. Also, additional optical devices, such as mirrors, may be positioned between the radiation source 106, light source 134, the eye 102, and the imaging devices 118, 150 to modify the direction of photons passing therebetween, thereby increasing flexibility in the placement of components within the aberration and corneal topography measurement apparatus of the present invention to accommodate housing and practical design constraints. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A method for measuring aberrations introduced by an eye and the topography of a surface of a cornea of the eye within the same device, said method comprising the steps of:
(a) directing a beam into the eye to produce a wavefront exiting the eye along a first path;
(b) projecting a pattern onto the surface of the cornea of the eye to produce a reflected pattern along said first path;
(c) directing said wavefront into a second path and said reflected pattern into a third path;
(d) determining from said wavefront aberrations introduced by the eye; and
(e) determining from said reflected pattern the topography of the surface of the cornea.

2. A method in accordance with claim 1, wherein said third path is in-line with said first path.

3. A method in accordance with claim 1, wherein said wavefront is characterized by a first frequency and said reflected pattern is characterized by a second frequency different from said first frequency.

4. A method in accordance with claim 3, wherein step (c) comprises directing said wavefront and said reflected pattern based on said first and second frequencies.

5. A method in accordance with claim 4, wherein step (c) is performed with a dichroic beam splitter.

6. A method in accordance with claim 1, wherein step (d) comprises:
passing said wavefront through a lenslet array to produce a plurality of images on an imaging plane; and
comparing the location of each of said plurality of images on said imaging plane to a corresponding reference location.

7. A method in accordance with claim 1, wherein step (d) is performed before step (e).

8. A method for measuring aberrations introduced by an eye and the topography of a surface of a cornea of the eye, said method comprising the steps of:
(a) directing a beam into the eye to produce a wavefront exiting the eye along a first path;
(b) projecting a pattern onto the surface of the cornea of the eye to produce a reflected pattern along said first path;
(c) differentiating said wavefront and said reflected pattern;
(d) determining from said wavefront aberrations introduced by the eye; and
(e) determining from said reflected pattern the topography of the surface of the cornea.

9. A method in accordance with claim 8, wherein said wavefront is characterized by a first frequency and said reflected pattern is characterized by a second frequency, and wherein step (c) is carried out by differentiating between said first and second frequencies.

10. An apparatus for measuring aberrations introduced by an eye and the topography of a surface of a cornea of the eye, said apparatus comprising:
a source for generating a beam capable of producing a wavefront exiting the eye;
a projector for projecting a pattern onto the cornea of the eye capable of being reflected by the cornea of the eye;
a combiner/separator for directing said wavefront and said reflected pattern;
a first imaging device for receiving said wavefront; and
a second imaging device for receiving said reflected pattern.

11. An apparatus in accordance with claim 10, further comprising a processor for processing information received from said first and second imaging devices.

12. An appararus in accordance with claim 10, wherein said combiner/separator is configured to direct sound wavefront on a first path and said reflected pattern on a second path.

13. An apparatus in accordance with claim 12, wherein said combiner/separator is further configured to direct said beam toward the eye.

14. The apparatus of claim 13, further comprising a common pathway, and wherein said beam directed toward the eye, said wavefront exiting the eye, and said reflected pattern as reflected by the eye travel along said common pathway.

15. An apparatus in accordance with claim 14, wherein said common pathway is collinear with said second path.

16. An apparatus in accordance with claim 14, wherein said common pathway extends through said pattern projector.

17. An apparatus in accordance with claim 10, wherein said combiner/separator is a dichroic beam splitter.

18. An apparatus in accordance with claim 17, wherein said dichroic beam splitter separates frequencies of light based on a selected pass wavelength.

19. An apparatus in accordance with claim 18, wherein said wavefront has a first wavelength greater than said selected pass wavelength and said reflected pattern has a second wavelength less that said selected pass wavelength.

20. An apparatus in accordance with claim 19, wherein said selected pass wavelength has a wavelength selected above about 700 nm.

21. An apparatus in accordance with claim 19, wherein said selected pass wavelength is about 720 nm, said first wavelength is above about 760 nm, and said second wavelength is below about 680 nm.

22. An apparatus in accordance with claim 10, wherein said projector comprises at least a Placido ring projector.

23. An apparatus in accordance with claim 10, wherein said projector includes a passageway for passing said beam, said wavefront, and said reflected pattern.

24. An apparatus in accordance with claim 10, wherein the apparatus is housed within a handheld device.
